(19) United States
(12) Patent Application Publication
(10) Pub. No.: US 2023/0149639 A1
(43) Pub. Date: May 18, 2023

(54) A SYRINGE, A METHOD FOR MANUFACTURING SUCH SYRINGES AND AN ASSEMBLY

(71) Applicant: Medtech Concept LLC, Lake Worth, FL (US)

(72) Inventors: Patrick MARTINSSON, ADT (MT); Ivar FRISCHER, Gothenberg (SE)

(21) Appl. No.: 17/919,587

(22) PCT Filed: Apr. 21, 2020
(86) PCT No.: PCT/US2020/029047

§ 371 Date: Oct. 18, 2022

(51) Int. Cl. A61M 5/50; A61M 5/315

(52) U.S. Cl. CPC A61M 5/5066; A61M 5/31515; A61M 2207/10

(57) ABSTRACT

A syringe, having a front portion carrying a hollow, axially extending needle, a container, running from said front portion and forming a container rear end portion, a plunger, extending and being movable axially in the container, the needle rear end being in fluid connection with the container and the plunger being arranged to suck liquid, e.g. a liquid comprising insulin, through the needle when drawn away from the needle and to discharge liquid through the needle when pushed towards the needle, the plunger having a piston portion, turned to the needle rear end, and a coupling portion arranged to co-act with the piston portion for moving the piston portion in the container.

### TECHNICAL FIELD

The present invention relates to a syringe according to the introductory portion of the attached claim 1. The present invention further relates to a method according to the attached claim 13 for manufacturing such syringes. Still further, the present invention relates to an assembly according to the attached claim 15.

### BACKGROUND

A general problem associated with syringes, especially those intended for self-injecting insulin into diabetes patients, is that they are quite expensive and also extensive and, thus, not easy to handle and to carry with you, e.g. in a pocket, a bathing suit and the like, and, thus, not easy to use discretely.

Yet another general problem associated with syringes, especially those intended for injecting substances, e.g. a liquid comprising insulin, into the human body, is that it is normally preferable that they are intended for single injection purposes, this being for contamination reasons. Thus, well known are the extremely serious problems associated with re-use of syringes as a source of spread of infections.

Further, there is a problem to design a convenient and practical housing for one or more syringes according to the present invention.

Still further, there is a problem to manufacture syringes according to the present invention in large numbers to a reasonable cost, the need of syringes being most extensive.

### PRIOR ART

Therefore, there are a great number of prior art syringes being designed to prevent re-use, these designs being, however, complicated, expensive to manufacture and complicated to mount.

The object of the present invention is to provide a syringe less associated with the problems related to prior art syringes and yet, in principle, avoiding re-use possibilities.

Further, an object of the present invention is to provide a design and method for manufacturing syringes according to the present invention in large numbers to a low cost.

### SUMMARY OF THE INVENTION

These and other objects are obtained by a syringe according to what is specified in independent claim 1 as well as by a method according to what is specified in independent claim 13 and an assembly according to independent claim 15.

Further objects are obtained by what is specified in the dependent claims.

According to preferred embodiments, the size of the syringe is quite limited so as to provide comparatively discrete injecting and comfortable carrying in even quite tight or tapered clothes. Thus, according preferred embodiments the syringe, with the plunger fully drawn out, has a length in the range of 30-80 millimeters, preferably in the range of 40-60 millimeters.

Further, according to preferred embodiments, the size of the syringe is adapted to the kind of liquid to be injected, e.g. the concentration of the active substance of the liquid, which means that a larger volume of liquid is desired and therefore a bigger size of the syringe is necessary for obtaining a desired effect of a certain injection.

According to preferred embodiments the rear end of the plunger has a conical flange portion, narrowing towards the plunger front end and forming an edge portion running around the plunger rear end. In this way gripping is promoted.

According to preferred embodiments the rear end of the container is formed with a countersink arrangement to receive the rear end of the plunger. In this way the rear end of the plunger is fitted in the end portion of the container in the discharged state of the syringe so that gripping of the plunger rear end, in practice, is prevented, the end portion having a convex configuration to provide a hard to grip shape.

According to preferred embodiments said coupling portion front has a convex rounded configuration and a rear, substantially cylindrical, support portion. In this way the introduction into said cavity as well as the coupling action and the movability in the cavity is promoted.

According to preferred embodiments the piston portion is made of a resilient material, such as a rubber based material or a polymeric material. In this way a liquid tight connection with the container as well as mounting on the coupling portion is promoted.

According to preferred embodiments the syringe is substantially axially symmetrical. This makes the syringe easy to handle and to manufacture.

According to preferred embodiments an end portion of the plunger is provided with a rearward open channel like recess intended to receive a mounting pin of a mounting assembly, the mounting pin being intended to support syringe parts during mounting of the syringe, whereby a very efficient mounting procedure may be obtained for a large number of syringes at a low cost.

According to preferred embodiments the front portion with the needle is covered and, thus, protected during handling and transportation by a potential user by a cover in the form of a sleeve with a front wall and an open rear end portion, the cover being designed to be removably applied to said front portion and held by internal friction against an internal ridge, the cover being made of a resilient material other than the material of the front portion. The cover has a cylindrical rear portion running from an outer stop flange, the rear portion being intended to fit in a syringe housing, and being held by an external ridge. In this way syringes may be handled liquid tightly disposed in said housing and handled liquid tightly protected by said cover when removed from said housing.

### BRIEF DESCRIPTION OF THE DRAWINGS

A better understanding of the present invention may be had by considering the following detailed description in conjunction with the attached drawings, in which like details are designated with like designations and in which:

FIG. 1 diagramatically shows in a) a syringe unit comprising a needle, a front portion and a container, in b) a needle cover, in c) a piston with a rear recess and in d) a plunger portion;

FIG. 2 shows two projections of a single syringe housing;

FIG. 3 shows two projections of a three syringe housing;

FIG. 4 shows, in cross section, a single syringe housing containing a syringe shown in a delivery state and, thus, not yet filled with liquid;

FIG. 5 shows, in cross section, a three syringe housing containing three syringes, the bottom one being fully discharged and the middle and the top ones being filled with liquid and ready for discharging said liquid;

FIG. 6 shows, in cross section, a mounting pin;

FIG. 7 a)-e) show five different stages of mounting of syringes in a mounting assembly, wherein five groups of four syringes held in a syringe row are shown to the left and, in different stages of mounting, five groups of four mounting pins arranged for performing different mounting steps are shown to the right;

FIG. 8 a)-e) show four different states of syringe use, a) showing a delivery state, b) showing a state wherein liquid being sucked into the syringe, c) showing a state wherein the syringe is ready for discharging said liquid, d) showing a state wherein the syringe is only partly filled with liquid and e) showing a discharged state; and

FIG. 9 schematically shows an assembly according to the present invention.

### DETAILED DESCRIPTION OF A PREFERRED EMBODIMENT

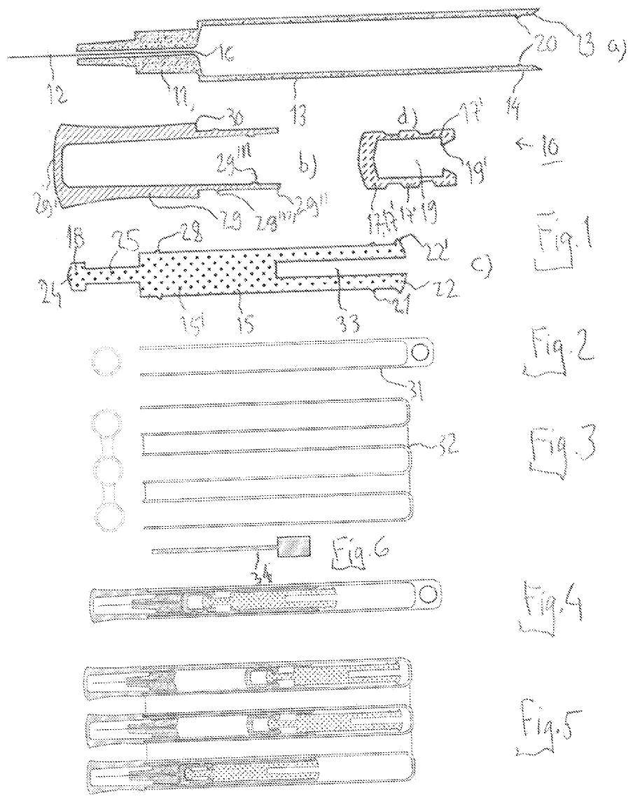

According to the embodiment shown in separate parts in FIG. 1a)-d), the syringe 10 comprises a front portion 11 carrying a hollow, axially extending needle 12 indicated in the Figs, a container 13, running from said front portion 11 and forming a container rear end portion 14. There are several different arrangements for applying said needle 12 at the front portion 11. According to one embodiment the needle is connected to a sleeve portion arranged to be force fit onto said front portion. According to another embodiment the needle is connected to a sleeve portion arranged to be screwed onto said front portion and thereby being fastened by a thread like arrangement.

The syringe further comprises a plunger 15, extending and being movable axially in the container 13, the needle rear end 16 being in fluid connection with the container and the plunger being arranged to suck liquid through the needle when drawn away from the needle and to discharge liquid through the needle when pushed towards the needle.

The plunger has a piston portion 17, turned to the needle rear end, and a coupling portion 18 arranged to co-act with the piston portion for moving the piston portion in the container. The piston portion and the plunger body 15′ comprising the coupling portion are separate parts, the piston portion being connected to the plunger body by means of the coupling portion.

Thus, the coupling portion is introduced into a cavity 19 in the piston portion and is movable axially in said cavity between a front position and a rear position, the plunger length in relation to the container length being adapted so that when the syringe is arranged in a delivery state, FIG. 8a), with the piston portion in a front position in the container and with the coupling portion in said rear position in said cavity, the rear end 15″ of the plunger is extending a certain distance beyond the rear end portion 14 of the container, thereby forming a plunger gripping portion 15′″.

Further, the container rear end portion 14 comprises an internal locking flange arrangement 20 and the plunger portion 15″ extending a certain distance beyond the container rear end portion comprises an outer locking flange arrangement 21, so that when the plunger in a discharged state, FIG. 8e), is pushed forward to the front position of the coupling portion in said cavity, the locking flange arrangement of the plunger is pushed forward, passing the locking flange arrangement of the container, thereby locking the plunger in the container in a non re-use state of the syringe.

According to preferred embodiments the rear end gripping portion 15′″ of the plunger has a flange portion 22 forming an edge portion 22′ of the plunger rear end.

According to preferred embodiments the rear end 14 of the container is formed with a countersink arrangement 23 to receive the rear end gripping portion 15′″ of the plunger in a discharged state of the syringe, thus making the end 15″ extremely hard or even impossible to grip, as can be seen in FIG. 8e)).

Further according to preferred embodiments, said coupling portion 18 has a front convex and rounded tip configuration 24, to promote introduction into said cavity and its coupling action for passing a rear stop flange 19′ of the piston portion, and a rear, substantially cylindrical, support portion 25 to pass into and out of said cavity.

Preferred is also that the piston portion is made of a resilient material, such as a rubber based material or polymeric material, the piston portion being axially longer than the inner diameter of the container in order to prevent tilting in the container. To promote low friction the front, rear and middle of the piston portion are designed as flanges 17′.

Further, according to preferred embodiments, the syringe is substantially axially symmetrical.

Further, the piston body 15′ front end, from which the coupling portion is extending, comprises a flange arrangement 28, which is intended to co-act with the flange arrangement 20 of the container when the plunger is pulled backwards and thus preventing the plunger from leaving the container.

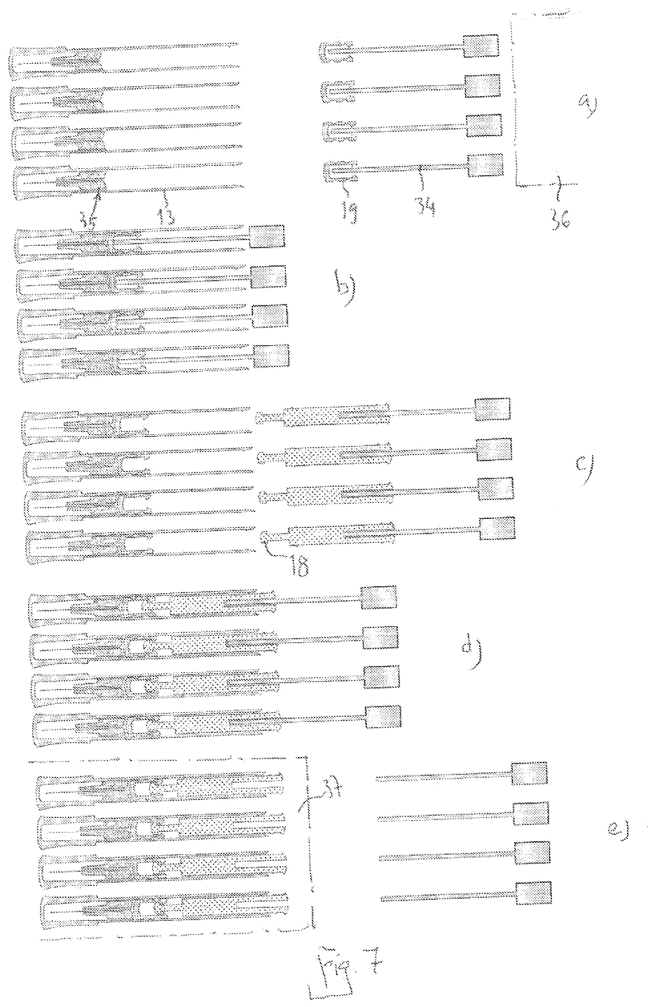

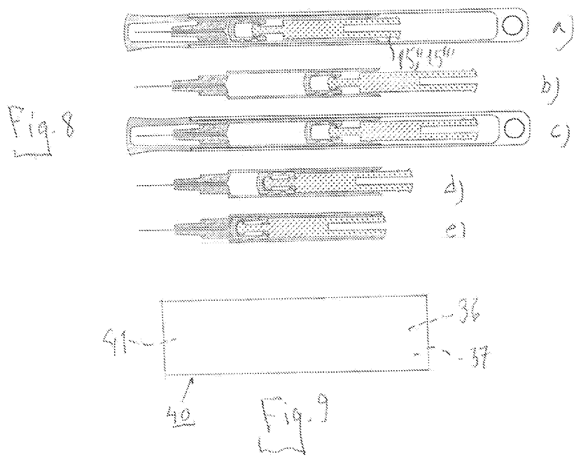

According to preferred embodiments the front portion with the needle is covered and, thus, protected during handling and transportation by a potential user by a cover 29 in the form of a sleeve with a front wall 29′ and an open rear end 29″ portion, the cover being designed to be removably applied to said front portion and held by internal friction against an internal ridge 29′″, the cover being made of a resilient material other than the material of the front portion. The cover has a cylindrical rear portion running from an outer stop flange 30, the rear portion being intended to fit in a syringe housing 31,32 as shown in FIG. 4 or 7, and being held by an external ridge 29″″.

According to preferred embodiments there are housings provided for one or more syringes for protection during transport, carrying and handling of the syringes, the housing for more than one, for example three, syringes providing a record for the number of syringes used for injection by the user, used syringes preferably being returned into the housing.

According to preferred embodiments an end of the plunger 15 is provided with a rearward open channel like recess 33, FIG. 1, intended to receive a mounting pin 34, FIG. 6, of a mounting assembly 40, the mounting assembly being, described later in association with FIG. 7, the mounting pin being intended to support syringe parts during mounting of the syringe.

The function of the syringe according to the present invention should to a considerable and sufficient extent have been made clear by the description given above.

Thus, in a delivery state, FIG. 8a), the syringe is arranged with the piston portion in a front position in the container and with the coupling portion 18 in said rear position in said cavity, the rear end 15″ of the plunger extending a certain distance beyond the rear end 14 of the container, thereby forming a plunger gripping portion 15′″.

To fill the syringe the needle is introduced into a liquid, e.g. a liquid comprising insulin, glycagon, a vaccine or other substances, the plunger is pulled backwards in the container, whereby the liquid is sucked into the container through the needle by the piston portion drawn by the coupling portion to fill the container to a desired extent.

To discharge liquid from the syringe to empty it through the needle, the plunger is pushed forward in the container. The coupling portion is pushed forward to the front position in said cavity and the locking flange arrangement of the plunger is pushed forward, passing the locking flange arrangement of the container, thereby locking the plunger in the container in a non re-use state of the syringe.

According to the preferred embodiments the countersink arrangement of the container rear end receives the rear end gripping portion (15′″) of the plunger, thus making it extremely difficult or impossible to draw the plunger backwards again to re-use the syringe.

An important part of the present invention is also a method for manufacturing syringes according to the present invention, the syringes, in general terms, having a front portion (11) carrying a hollow, axially extending needle (12), a container (13), running from said front portion and forming a container rear end portion (14), a plunger (15), extending and being movable axially in the container, the needle rear end (16) being in fluid connection with the container and the plunger being arranged to suck liquid, e.g. a liquid comprising insulin, glycagon, a vaccine or other substances, through the needle when drawn away from the needle and to discharge liquid through the needle when pushed towards the needle, the plunger having a piston portion (17), turned to the needle rear end, and a coupling portion (18) arranged to co-act with the piston portion for moving the piston portion in the container.

According to preferred embodiments the method comprises the steps of
providing the plunger 15 with a channel like, axially running recess 33, FIG. 1c), having a rear opening and being intended to receive a mounting pin 34 for supporting syringe parts 15,19 during mounting of the syringe;
providing a unit 35 comprising said container 13 with the needle 12 arranged in the front portion 11, FIG. 7a) and holding said unit in a mounting position and carrying the piston portion 19 by means of the mounting pin, FIG. 7a);
introducing the piston portion 19 in the container by means of the mounting pin, FIG. 7b);
carrying the plunger by means of the mounting pin, FIG. 7c);
introducing the coupling portion 18 of the plunger into the piston portion for co-action for moving the piston portion forward and backwards in the container by means of the mounting pin, FIG. 7d), and
bringing the syringe into a delivery state by removing the mounting pin, FIG. 7e).

The steps are illustrated in Figs. a)-e).

According to preferred embodiments of a mounting assembly 40 according to the present invention, a desired number of mounting pins are arranged on a mounting frame 36, movable to and from syringe units 35 held and carried by a syringe unit frame 37 for performing the necessary steps of the method according to the present invention. Of course the four syringes being mounted in FIG. 7 is only an example, the number being intended to be much greater.

Above the present invention has been described in association with preferred embodiments and examples. Of course, further embodiments as well as minor changes and additional details may be imagined without departing from the basic inventive idea of the present invention.

Further, according to preferred embodiments, the container and the plunger are made of polymeric material.

Thus, the present invention is not limited to the embodiments shown but may be varied within the scope of the attached claims.

1-15. (canceled)

16. A syringe, having a front portion carrying a hollow, axially extending needle, a container, running from said front portion and forming a container rear end portion, a plunger, extending and being movable axially in the container, the needle rear end being in fluid connection with the container and the plunger being arranged to suck liquid, e.g. a liquid comprising insulin, glycagon, a vaccine or other substances, through the needle when drawn away from the needle and to discharge liquid through the needle when pushed towards the needle, the plunger having a piston portion, turned to the needle rear end, and a coupling portion arranged to co-act with the piston portion for moving the piston portion in the container, wherein the coupling portion is introduced into a cavity in the piston portion and is movable axially in said cavity between a front position and a rear position, the plunger length in relation to the container length being adapted so that when the syringe is arranged in a delivery state with the piston portion in a front position in the container and with the coupling portion in said rear position in said cavity, the rear end of the plunger is extending a certain distance beyond the rear end of the container, thereby forming a plunger gripping portion, and wherein the container rear end portion comprises an internal locking flange arrangement and the plunger portion extending a certain distance beyond the container rear end portion comprises an outer locking flange arrangement, so that when the plunger body, comprising the coupling portion, in a discharge state is pushed forward to the front position of the coupling portion in said cavity, the locking flange arrangement of the plunger is pushed forward, passing the locking flange arrangement of the container, thereby locking the plunger in the container in a non re-use state of the syringe.

17. The syringe according to claim 16, wherein said gripping portion of the plunger has a flange portion forming an edge portion of the plunger rear end, the end of the gripping portion having a convex shape extending from the edge portion to prevent gripping.

18. The syringe according to claim 16, wherein the rear end of the container is formed with a countersink arrangement to receive the rear end gripping portion of the plunger and to cover said edge portion.

19. The syringe according to claim 16, wherein said coupling portion has a convex tip configuration, to promote introduction into said cavity and its coupling action, and a rear, preferably substantially cylindrical, support portion.

20. The syringe according to claim 16, wherein the piston portion having a convex front end for promoting introduction into the container, a generally cylindrical configuration and, preferably, flange portions for sealingly contacting the inner surface of the container.

21. The syringe according to claim 16, wherein the piston portion is made of a resilient material, such as a rubber based material or a polymeric material.

22. The syringe according to claim 16, wherein the syringe is substantially axially symmetrical.

23. The syringe according to claim 16, wherein an end portion of the plunger is provided with a rearward open channel like recess intended to receive a mounting pin of a mounting assembly, the mounting pin being intended to support syringe parts during mounting of the syringe.

24. The syringe according to claim 16, wherein the front portion with the needle is covered and, thus, protected during handling and transportation by a potential user, by a cover in the form of a sleeve with a front wall and an open rear end portion, the cover being designed to be removably applied to said front portion and held by internal friction between an internal ridge and said front portion, the cover being made of a resilient material other than the material of the front portion.

25. The syringe according to claim 24, wherein the cover has a cylindrical rear portion running from an outer stop flange, the rear portion being intended to fit in a syringe housing.

26. The syringe according to claim 24, wherein the cover provides a liquid tight connection between the syringe front portion and the cover as well as between the syringe housing and the cover by means of internal and external ridges.

27. The syringe according to claim 16, wherein the syringe, with the piston drawn out, has a length in the range of 30-80 millimeters, preferably in the range of 40-60 millimeters.

28. A method for manufacturing a syringe, having a front portion carrying a hollow, axially extending needle, a container, running from said front portion and forming a container rear end portion, a plunger, extending and being movable axially in the container, the needle rear end being in fluid connection with the container and the plunger being arranged to suck liquid, e.g. a liquid comprising insulin, glycagon, a vaccine or other substances, through the needle when drawn away from the needle and to discharge liquid through the needle when pushed towards the needle, the plunger having a piston portion, turned to the needle rear end, and a coupling portion arranged to co-act with the piston portion for moving the piston portion in the container, the method comprising the steps of:
providing the plunger with a channel like, axially running recess, having a rear opening and being intended to receive a mounting pin;
providing a unit comprising said container with the needle arranged in the front portion, and holding said unit in a mounting position and carrying the piston portion by means of the mounting pin;
introducing the piston portion in the container by means of the mounting pin;
carrying the plunger by means of the mounting pin;
introducing the coupling portion of the plunger into the piston portion for co action for moving the piston portion forward and backwards in the container by means of the mounting pin; and
bringing the syringe into a delivery state by removing the mounting pin.

29. The method according to claim 28, wherein said unit comprises a cover applied to the syringe front portion.

30. A mounting assembly for performing the method according to claim 28, wherein means are provided for carrying a desired number of mounting pins on a mounting frame and to move said frame to and from syringe units held and carried by a syringe unit frame and for performing the steps of the method.
